(19) United States
(12) Patent Application Publication
(10) Pub. No.: US 2025/0049966 A1
(43) Pub. Date: Feb. 13, 2025

(54) SMALL MOLECULE TRACER FOR IMAGING ALPHA-SYNUCLEIN AGGREGATES

(71) Applicant: Fudan University, Shanghai (CN)

(72) Inventors: Yong CHU, Shanghai (CN); Jian WANG, Shanghai (CN); Jiang BIAN, Shanghai (CN); Yiqi LIU, Shanghai (CN); Xin LIN, Shanghai (CN); Chenyang QIU, Shanghai (CN); Jie HE, Shanghai (CN); Deyong YE, Shanghai (CN)

(21) Appl. No.: 18/718,239

(22) PCT Filed: Dec. 12, 2022
(86) PCT No.: PCT/CN2022/138356

§ 371 Date: Jun. 10, 2024

(30) Foreign Application Priority Data
Dec. 13, 2021 (CN) 202111521713.9

(51) Int. Cl. A61K 51/04; C07D 413/04; C07D 413/14

(52) U.S. Cl. CPC A61K 51/0455; A61K 51/0459; C07D 413/04; C07D 413/14

(57) ABSTRACT

The invention discloses a type of compound that can specifically bind to α-synuclein aggregates represented by Formula I, a radio-labelled compound thereof, a preparation method, and its use. The compound can be used as a tracer for optical imaging of α-synuclein aggregates in biological samples or in vivo (such as the brain). After radio-labelled, the compound of the invention can be used as a radio imaging tracer for PET, SPECT, and other imaging techniques to realize the detection of α-synuclein lesions by non-invasive visualization in vivo (such as the brain).

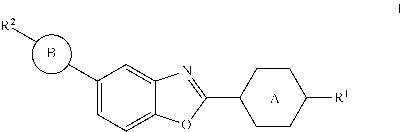

### FIELD OF THE INVENTION

The present invention relates to novel compounds that are useful as tracers of α-synuclein aggregates and their use in the imaging of α-synuclein.

### BACKGROUND OF THE INVENTION

α-synuclein (α-Syn) lesions are an important pathogenesis of neurodegenerative diseases (Vekrellis, 2010). The abnormal aggregation of α-synuclein and the formation of Lewy bodies and Lewy neurites with it as main components are the important pathological features and pathogenic factors of various neurodegenerative diseases including Parkinson's disease (PD), Parkinson's disease dementia (PDD), dementia with Lewy body (DLB), and multiple system atrophy (MSA). The process from the formation of α-synuclein deposition to the appearance of clinical symptoms is relatively long, usually lasting several years or even more than ten years, and it is too late to intervene when the patient has developed clinical symptoms. Early clinical intervention is very important to delay the progression of the disease and improve the quality of life and prognosis of patients. Therefore, the development of reliable early detection methods is the key point for the early diagnosis, prevention, and treatment of neurodegenerative diseases. At the same time, regulating the aggregation process of α-synuclein is also an important strategy for the treatment of these neurological diseases.

Due to its important role in the pathogenesis and progression of various neurodegenerative diseases, α-synuclein has become an important biomarker for early diagnosis of these diseases and an important target for drug therapy. However, the detection of α-synuclein aggregates currently can only be based on histological analysis of autopsy materials, and fail to make non-invasive detection in vivo. Molecular imaging is the best way to solve this problem.

Molecular imaging is based on the specific binding of molecular tracer (e.g., radioactive tracer, fluorescent tracer, etc.) to biomarkers (e.g., receptors, enzymes, ion channels, misfolded proteins), which are visualized by PET, SPECT, NMR, near-infrared, or other methods to provide diagnostic information in vivo. To realize molecular imaging, the key is to get a small molecule compound that can bind to a given molecular target as the imaging tracer. Since pathological changes of α-synuclein, Aβ, and Tau proteins are often co-deposited in neurodegenerative human brains, imaging tracers of the specific protein must have not only a strong affinity for the target protein aggregate but also a high selectivity for abnormal accumulations of other proteins to achieve selective imaging. To date, few small molecule tracers have been reported that can visualize α-synuclein deposition in the brain of patients.

### DISCLOSURE OF INVENTION

The purpose of the present invention is to provide a small molecule tracer capable of imaging α-synuclein aggregates and a radiographic labelled small molecule tracer for imaging diagnosis of diseases related to α-synuclein accumulation. The tracer of the invention can bind and image α-synuclein lesions in the brain of the patient, and after radio-labelled the tracer can be used by PET and SPECT to realize the non-invasive visual detection of α-synuclein in vivo, which can provide early diagnosis, disease monitoring, and drug efficacy evaluation for patients with neurodegenerative diseases such as Parkinson's disease, dementia with Lewy body and multiple system atrophy.

For the above purposes, the present invention provides compounds represented by Formula I, salts thereof, and solvates thereof. The compound has a strong affinity for α-synuclein aggregates and good blood-brain barrier permeability, especially strong binding/staining of Lewy bodies and Lewy neurites but weak for Aβ and Tau in patient brain tissue, indicating good selectivity. Therefore, the compound of the invention can be used as a fluorescent imaging tracer of α-synuclein aggregates, or as a radiometric imaging tracer for PET, SPECT, and other imaging technologies after radio-labelled to perform non-invasive visual imaging of the pathology of α-synuclein in vivo (such as the brain).

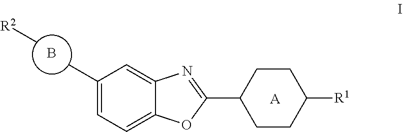

wherein,
Ring A is selected from phenyl, pyridinyl, pyrimidine group;
R¹ is selected from 4-6 membered nitrogen-containing cycloalkyl, amino group substituted with N, N-diC₁₋₃ alkyl, C₁₋₃ alkoxyl, nitro, halogen;
Ring B is selected from pyridinyl, piperazinyl, piperazinone group;
R² is selected from hydrogen, halogen, hydroxyl, C₁₋₃ alkyl, C₁₋₄ alkoxyl, and halogenated C₁₋₄ alkoxyl;

Wherein, the halogen is selected from fluorine, chlorine, bromine, or iodine;

Wherein, one or more atoms of the compound of Formula I are the radioisotopes of that atom, preferably taken from ¹¹C, ¹³N, ¹⁵O, ¹⁸F, ⁷⁶Br, ¹²³I, ¹²⁵I, and ¹³¹I.

The invention also provides precursor compounds for the preparation of radiolabelled formula I compounds, whose structures are shown as follows:

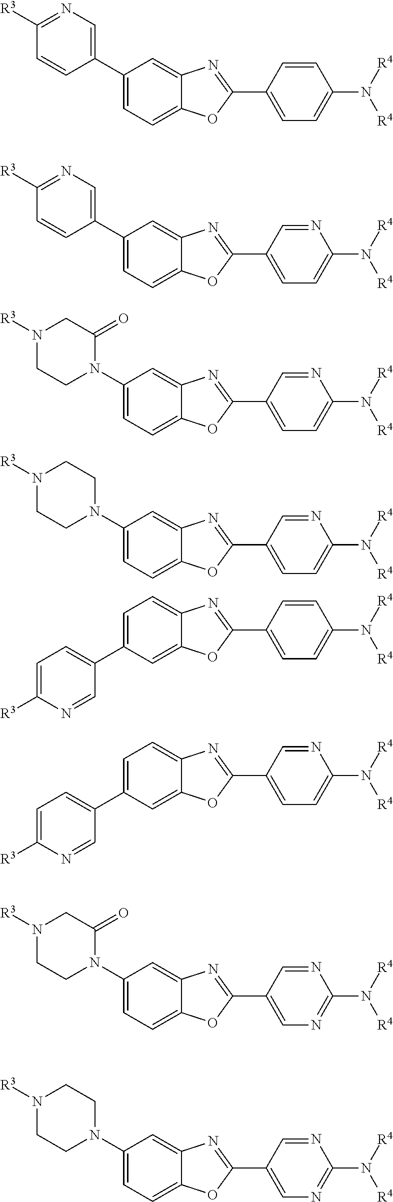

Wherein, R³ is independently selected from hydroxyl, fluorine, bromine, iodine, nitro, borate group, TsO-(CH₂)ₘ—, MsO-(CH₂)ₘ—, wherein m is an integer ranging from 0 to 4. R⁴ is hydrogen, C₁₋₃ alkyl, 4-6 membered nitrogen-containing cycloalkyl by cyclization of NR⁴R⁴. One or more atoms of the Formula I compound can be labelled as a radionuclide of that atom by the precursor compound. Accordingly, the present invention also provides radiolabelled formula I compounds, preferably selected from the following structures:

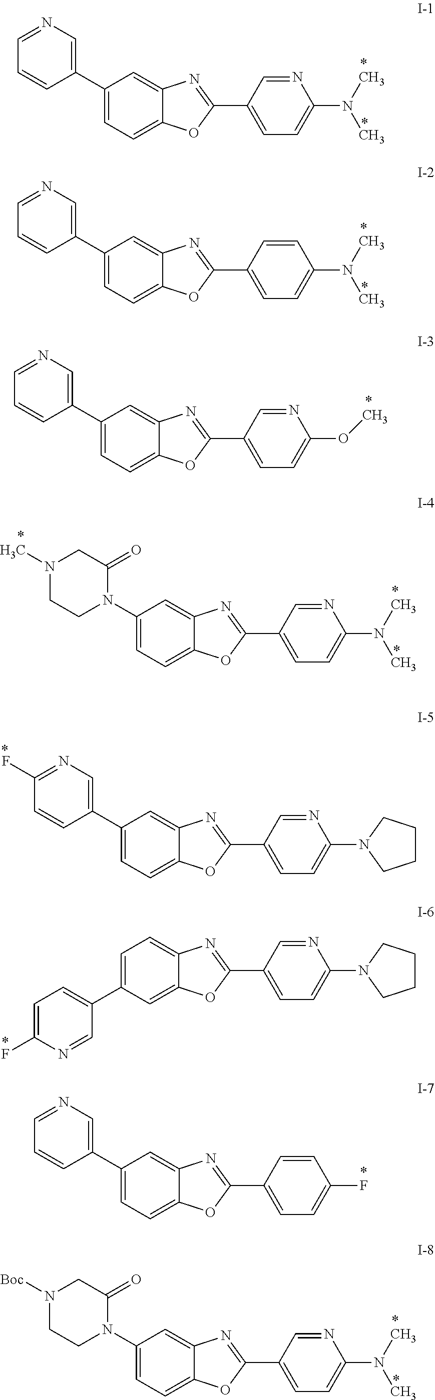

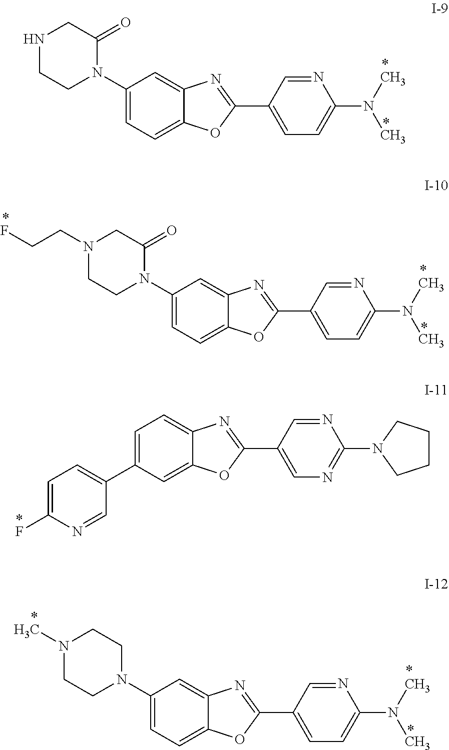

Wherein one of the atoms marked with * is a radioisotope of that atom at least.

The invention also provides the use of Formula I compounds that can specifically bind to α-synuclein aggregates. The compound exhibits self-fluorescence and can be used as a tracer for fluorescence imaging. As one or more atoms of the compound are replaced by radioactive atoms, they can be used as radiometric tracers for various imaging techniques. For example, when one or more fluorine or carbon atoms of the compound are replaced by radionuclides ¹⁸F or ¹¹C, they can be used as radiometric tracers for imaging of Positron Emission Tomography (PET), or for the preparation of the imaging tracers or the preparation of a composition comprising the imaging tracer. These imaging tracers can be used to detect neurological diseases associated with α-synuclein misfolding and aggregation, to screen for therapeutic or preventive drugs for diseases associated with α-synuclein aggregates in the brain, or to quantify or determine the accumulation of α-synuclein aggregates in the brain.

### The Beneficial Effects of the Invention:

Positron Emission Tomography (PET) and Single-Photon Emission Computed Tomography (SPECT) are the most advanced non-invasive 3D imaging techniques. The use of PET and SPECT radiotracer that binds specifically to a given biologic target can provide in vivo real-time diagnostic information closest to pathology to prove and quantify pathophysiological changes related to the disease, and the most powerful tools for early clinical diagnosis, disease progression monitoring, and therapeutic drug development. Radionuclides used for PET generally include ¹¹C, ¹³N, ¹⁵O, and ¹⁸F, with radioactive half-lives of 20, 10, 2, and 110 minutes, respectively. ¹⁸F is usually the best choice as a radionuclide for PET because it has the longest half-life and so is the most convenient to use. In addition, ^{99m}Tc, ¹²³I, ¹³¹I, and ¹¹¹In are the radionuclides most commonly used in SPECT. In principle, these nuclides can be used to replace any corresponding non-radioactive isotope atom in the tracers to make it radioactive.

Therefore, when a radionuclide is labelled to a specific tracer of α-synuclein aggregates, it can be used as a tracer for autoradiography in vitro or PET/SPECT imaging in vivo to achieve visualization of pathological α-synuclein. It has greatly facilitated the diagnosis, management, mechanism research, and development of therapeutic drugs for neurological disorders associated with α-synuclein misfolding and aggregation. The key to realizing imaging is to find tracers with high affinity and selectivity to α-synuclein and to label them with radionuclides as imaging tracers for PET and SPECT.

The present invention provides a novel type of compounds that have strong affinity and high specificity for binding of α-synuclein aggregates and can cross the blood-brain barrier. These compounds exhibit autofluorescence and can highly specific binding and clear staining of Lewy bodies and Lewy neurites in patient brain tissue (the main component of which is α-synuclein aggregates). Thus the compounds can be used as fluorescent imaging tracers for α-synuclein. As one or more fluorine or carbon atoms of said compounds in the present invention are replaced with radionuclides ¹⁸F or ¹¹C, they can be used as radio-imaging tracers of autoradiography or PET for imaging α-synuclein aggregates in vitro or in vivo, especially in the brain. When halogen atoms in the compound of the invention are replaced with radioactive isotopes or other acceptable nuclides, they can be used as tracers for SPECT to image α-synuclein aggregates.

The invention also provides processes for the preparation of Formula I compounds and radio-labelled compounds thereof, as well as precursor compounds for the preparation of radio-labelled compounds and preparation methods thereof. Further, the invention also provides methods of diagnostic imaging and quantifying or determining α-synuclein accumulation in the brain, as well as drug screening for preventing or treating α-synuclein accumulation diseases by Formula I compounds or their compositions thereof.

### A BRIEF DESCRIPTION OF THE FIGURES

FIG. 1 is a fluorescence microscope photograph of the tracer of the invention to stain the brain slice of a patient with Lewy body dementia (DLB). White arrows indicate Lewy bodies (left) or Lewy neurites (right). The result shows that the tracer can strongly bind to the α-synuclein aggregates in the patient's brain.

FIG. 2 is a fluorescence microscope photograph of the tracer of the invention to stain the brain slice of a patient with Alzheimer's disease (AD). The white arrow represents the original Aβ plaque. The result shows that the tracer has good target selectivity in the patient's brain with very weak or even no binding to the Aβ and Tau lesions.

### The Best Way to Realize the Invention

In this description, “α-synuclein accumulation disease” refers to diseases in which α-synuclein is abnormally folded and accumulated in the brain, including but not limited to Parkinson's disease (PD), Parkinson's disease dementia (PDD), multiple system atrophy (MSA), Lewy body dementia (DLB), etc. The present invention provides the Formula I compound, its salt or solvate thereof as an imaging tracer to visualize α-synuclein in vivo or in vitro to give diagnostic and evaluation information for these diseases.

In the present invention, the tracer that can be used for imaging α-synuclein accumulation is represented by general Formula I, or its salts and solvates thereof. The preferred compounds are I-1˜I-11. In particular, I-4, I-5, and I-6 can well label the α-synuclein in Lewy bodies and Lewy neurites from the DLB patient's brain tissues, and very weak binding to Aβ and no binding to Tau in the brain tissue of patients with Alzheimer's disease (AD), which shows good specificity.

The invention also includes salts of Formula I compounds which nitrogen atoms or other groups can be used to form the pharmaceutically acceptable salts.

Any chemical formula given in the present invention is also intended to represent a form of the compound with isotopic labeling. The isotopically labelled compounds have the structure shown in Formula I, differing only in that one or more of the atoms are replaced by its radioisotope. Isotopes that may be incorporated into the compounds of the invention include isotopes of hydrogen, carbon, nitrogen, oxygen, fluorine, chlorine, and iodine, such as ²H, ³H, ¹¹C, ¹³C, ¹⁴C, ¹⁵N, ¹⁸O, ¹⁷O, ³⁵S, ¹⁸F, ³⁶Cl, ¹²³I, ¹²⁵I, and ¹³¹I, respectively. Substituting with heavier isotopes (such as deuterium, 2H) can provide certain advantages arising from greater metabolic stability (such as increased in vivo half-life or reduced dose requirements). Substitution with 2H can be used specifically to prevent the formation of unwanted radio metabolites or to block radio-defluorination. When used to label the compound of the invention, positron radionuclides like ¹¹C, ¹³N, ¹⁵O, and ¹⁸F are preferred for PET imaging, in which ¹⁸F is the most preferred and ¹¹C next. Additionally, ¹²³I is preferred among y radionuclides for SPECT imaging.

The invention also includes radio-labelled Formula I compounds. In theory, any sites at the compound of Formula I can be labelled by a radionuclide, but it is preferable to replace the halogen, nitro, or label the alkyl group as shown in the examples. For example, when the compounds of the present invention are labelled with ¹⁸F, any location of the compound can be labelled, preferably replacing nitro or fluorine atoms.

The radiolabelled compounds of the present invention and their precursors required for a label can generally be prepared by conventional processes and schemes disclosed in examples, or by the following preparation methods (substituting non-isotopic labeling reagents by readily available isotopic labeling reagents). Some methods have been reported to label ¹¹C, ¹⁵N, ¹⁸O, ¹⁸F, or other isotopes to compounds (Angew. Chem. Int. Ed. Miller, Philip W, 2008, 47, 8998-9033; Peter J. H. Scott, 2009, 48, 6001-6004; Chem. Rev., Sean Preshlock, 2016, 116, 719-766; Frederic Dolle, Fluorine-18 chemistry for molecular imaging with positron emission tomography. Fluorine and Health: Molecular Imaging, Biomedical Materials and Pharmaceuticals (Tressaud, A. Haufe, G.), 2008, pp. 3-66, Elsevier). The Formula I compounds with radionuclide-labelled can be used as PET or SPECT tracers for imaging α-synuclein accumulations in vivo.

The present invention also provides precursor compounds for preparing radionuclide-labelled Formula I compounds. A person skilled in the art may design and synthesize the precursor compound according to the structure shown in the invention. That is, the precursor compound can be obtained by structurally modifying the commercially available compound or the compound of the present invention.

The radio-labelled compounds of the invention can be prepared by different precursor compounds. Typically, the labelled sites of precursors contain hydroxyl or nitro, bromine, iodine, borate, or other leaving groups (such as MsO-, TsO-, etc.), which can be substituted with ¹¹C or ¹⁸F, respectively. In particular, the precursor compound containing the hydroxyl can be obtained by removing the methyl from the methoxyl, and then directly labelled with ¹¹C, or use the brominated alkanes labelled with ¹⁸F, like ¹⁸F—CH₂CH₂—Br with oxo-alkylation reaction to produce ¹⁸F—CH₂CH₂—O— substituted compounds to achieve radioactive labelled. Similarly, the precursor compound may also contain nitro, bromine, iodine, borate, and leaving groups of TsO- or MsO-, which can be replaced by ¹⁸F by well-known methods. In the synthesis of compounds such as I-5, 1-6, and I-10, the position to be labelled in the precursor compound is preferentially converted to easily leaving groups of TsO- or MsO-.

Generally, the nuclides used for labeling are produced by cyclotrons, and a skilled person in the field may choose the appropriate method and instrument according to the nuclide to be manufactured. The methods of labeling are known in the field and mainly include chemical synthesis, isotope exchange, and biosynthesis. The radio-labelled compound of the invention can be administered locally or systematically to the patient, and after sufficient time of binding and dissociation with α-synuclein, the detection site can be visualized by PET and SPECT. The administration can be subcutaneous, abdominal, intravenous, arterial or spinal fluid injection or infusion, or oral, with full attention to the patient's exposure dose, depending on factors like the type of disease, the nuclide labelled, the compound used, the patient's condition, the difference of test site, etc.

The present invention also provides compositions for imaging diagnosis of α-synuclein accumulation diseases, which comprise compounds of the present invention, pharmaceutically acceptable salts thereof, or solvates thereof, and pharmaceutically acceptable carriers. The preferred composition comprises the labelled compounds of the present invention, wherein labeling with radionuclides (in particular positron radionuclides ¹¹C, ¹³N, ¹⁵O, ¹⁸F, etc.) is preferred for in vivo imaging diagnostics. Depending on its use, the compound or its composition thereof is preferably a form of injection for application. Therefore, pharmaceutically acceptable carriers are preferred to be liquid, including (but not limited to) aqueous solvents (such as potassium phosphate buffers, salt water, Ringer's solution, and distilled water) or anhydrous solvents (such as polyethylene glycol, vegetable oil, ethanol, glycerin, dimethyl sulfoxide, and propylene glycol). The proportion of the carrier and the compound of the invention can be appropriately varied, depending on the site of action, detection means, etc. In addition, the composition thereof may include commonly used antimicrobials (such as antibiotics, etc.), local anesthetics (such as procaine hydrochloride, ibucaine hydrochloride, etc.), buffers (such as trihydrochloric acid buffers, HEPES buffers, etc.), osmotic pressure regulators (such as glucose, sorbitol, sodium chloride, etc.).

The compounds of the invention include labelled or un-labelled ones which can be labelled before use by the methods described above.

The compound of the invention can bind to α-synuclein highly and specifically and therefore be used for staining and quantification of α-synuclein in vitro through labelled or un-labelled compounds. For example, due to their self-fluorescence, the compounds can be directly used to stain α-synuclein in a specimen and observe the fluorescence results by laser confocal or fluorescence microscopy or colorimetric to quantify α-synuclein. A scintillation counter is used for the quantification of α-synuclein after radiolabeling of the compounds. The early pathological of synuclein diseases such as Parkinson's disease (PD), dementia with Lewy body (DLB), multiple system atrophy (MSA), etc., is the formation of Lewy bodies, in which the main component is the abnormal accumulation of α-synuclein, and so the detection of Lewy bodies can provide the early onset information of these diseases. Because the compound of the invention can stain Lewy bodies and Lewy neurites, it can be used to study the pathological mechanism and the diagnosis of patients in the clinic. Staining brain sections using compounds of the invention can be performed by common methods.

As mentioned above, the compounds of the present invention, i.e. the compounds shown in general Formula I and their salts or solvates thereof, can be used as imaging tracers for α-synuclein accumulations, preferably radionuclide labelled imaging tracers.

Therefore, the present invention provides:

Formula I compounds, and their pharmaceutically acceptable salts or solvates thereof, are used as tracers for imaging α-synuclein accumulations:

Optical and radioactive tracers for imaging diagnosis of α-synuclein accumulation diseases, in particular, positron radionuclide labelled imaging tracers:

The precursor compounds for the preparation of radioisotopically labelled Formula I compounds:

A composition for the imaging diagnosis of α-synuclein accumulation diseases comprising a compound of Formula I, or its pharmaceutically acceptable salt or solvate thereof, and pharmaceutically acceptable carrier:

The use of compounds of Formula I, their pharmaceutically acceptable salts, and solvates thereof for the imaging diagnosis of α-synuclein accumulation diseases:

The use of compounds of Formula I, their pharmaceutically acceptable salts, and solvates thereof in the production of compositions for the imaging diagnosis of α-synuclein accumulation diseases.

In addition, the present invention provides:

Methods for detection/staining of α-synuclein aggregates, and Lewy bodies and Lewy neurites in patients' brains, which can be used to provide information for early diagnosis and assessment of progression of diseases with α-synuclein accumulation:

Methods for quantifying or determining the accumulation of α-synuclein in the brain:

Drug screening methods for the prevention and/or treatment of α-synuclein accumulation diseases:

In all the above methods, it is used of Formula I compounds, or their pharmaceutically acceptable salts or solvates thereof with pharmaceutically acceptable carriers.

In the following, substituents of Formula I compounds are explained, and salts, solvates, and derivatives of Formula I compounds are explained, as well as the labeling methods.

### Definition

Unless otherwise indicated, the meaning and scope of the terms of the present invention are described and limited as defined below.

The terms “compound of Formula I”, “Formula I compounds”, or “compound of the present invention” refer to any compound selected from a class of compounds represented by Formula I, including its stereoisomers, cis-trans isomers, tautomers, solvates, and salts (e.g., medicinal salts).

Unless otherwise specified, the use of “or” or “and” means “and/or”.

When indicating the number of substituents, the term “one or more” means the number of substituents from one to the largest chemically possible number, i.e., substituting one hydrogen to all hydrogens by a substituent.

The term “substituent” refers to an atom or group of atoms that replaces the hydrogen atoms on the parent molecule.

The term “halogen” refers to fluorine (F), chlorine (Cl), bromine (Br), and iodine (I).

The term “TsO-” means structure as

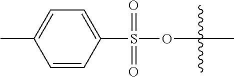 [SMILES: *OS(=O)(=O)c1ccc(C)cc1]

and “MsO-” means structure as

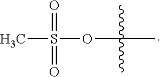 [SMILES: *OS(C)(=O)=O]

The term “C₁₋₄ alkoxyl” denotes the group of the formula —O—R′, wherein R′ refers to a univalent straight or branched saturated alkyl group containing 1 to 4 carbon atoms, for example, methoxy.

The term “halogenated C₁₋₄ alkoxyl” denotes an alkoxy group in which one or more hydrogen atoms have been replaced by the same or different halogen atoms (especially fluorine atoms), for example, 1-fluoroethoxy.

The term “C₁₋₃ alkyl” refers to univalent linear or branched-chain saturated hydrocarbon groups with 1 to 3 carbon atoms, for example, methyl.

The term “aromatic” is denoted by the conventional concept of aromaticity defined in the literature, especially IUPAC-Directory of Chemical Terms, 2nd Edition, A. D. McNaught & A. Wilkinson. Blackwell Scientific Publications, Oxford (1997).

The term “pharmaceutically acceptable salt” refers to salt that is not harmful to mammals, especially humans. Pharmaceutically acceptable salts can be formed by using non-toxic acids or bases, including inorganic or organic acids or bases, which include metal salts formed from aluminum, calcium, lithium, magnesium, potassium, sodium and zinc: Or with lysine, N, N′-dibenzyl ethylenediamine, chloroprocaine, choline, diethanolamine, ethylenediamine, meglumine (that is, N-methylglucamine) and procaine and other organic salts. In addition, the salts include acid-addition salts and alkali-addition salts.

The term “pharmaceutically acceptable carrier” means a saline solution, a pharmaceutically acceptable material, composition, or excipient such as a liquid or solid filler, diluent, solvent, or encapsulation material. Pharmaceutically acceptable carriers include but are not limited to water, salt solution, saline or phosphate buffered salt solution (PBS), sodium chloride injection, Ringer's injection, glucose injection, sterile water injection, glucose, and lactate Ringer's injection.

The term “solvate” refers to a solvent-containing compound formed by the association of one or more solvent molecules with the compound. For example, it may contain one solvate, two solvates, three solvates, and four solvates. In addition, solvates include hydrates.

The term “hydrate” refers to a compound or its salts containing water bound by non-covalent intermolecular forces, the amount of water contained may be stoichiometric or non-stoichiometric. For example, it contains monohydrate, dihydrate, trihydrate, and tetrahydrate.

### [Tracer of α-Synuclein Aggregates]

The present invention provides a tracer of α-synuclein aggregates (hereinafter also referred to as a tracer), i.e. a compound of Formula I showed below, a pharmaceutically acceptable salt thereof, or a solvate thereof.

In addition, the Formula I compounds shown below have spontaneous fluorescence, wherein one or more atoms in the compounds may be a radioactive isotope of the atom. Thus, the compounds of the invention can be used as small molecule tracers for optical imaging of α-synuclein aggregates accumulation, or for radio-imaging α-synuclein aggregates by PET and SPECT.

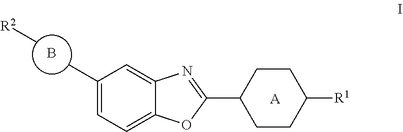

wherein,
Ring A is selected from phenyl, pyridinyl, pyrimidine group;
R¹ is selected from 4-6 membered nitrogen-containing cycloalkyl, amino group substituted with N, N-diC₁₋₃ alkyl, C₁₋₃ alkoxyl, nitro, halogen;
Ring B is selected from pyridinyl, piperazinyl, piperazinone group;
R² is selected from hydrogen, halogen, hydroxyl, C₁₋₃ alkyl, C₁₋₄ alkoxyl, and halogenated C₁₋₄ alkoxyl:

Wherein, the halogen is selected from fluorine, chlorine, bromine, or iodine; Wherein, one or more atoms of the compound of Formula I are the radioisotopes of that atom, preferably taken from ¹¹C, ¹³N, ¹⁵O, ¹⁸F, ⁷⁶Br, ¹²³I, ¹²⁵I, and ¹³¹I. For example, the following compounds may be cited as examples of Formula I compounds shown below:

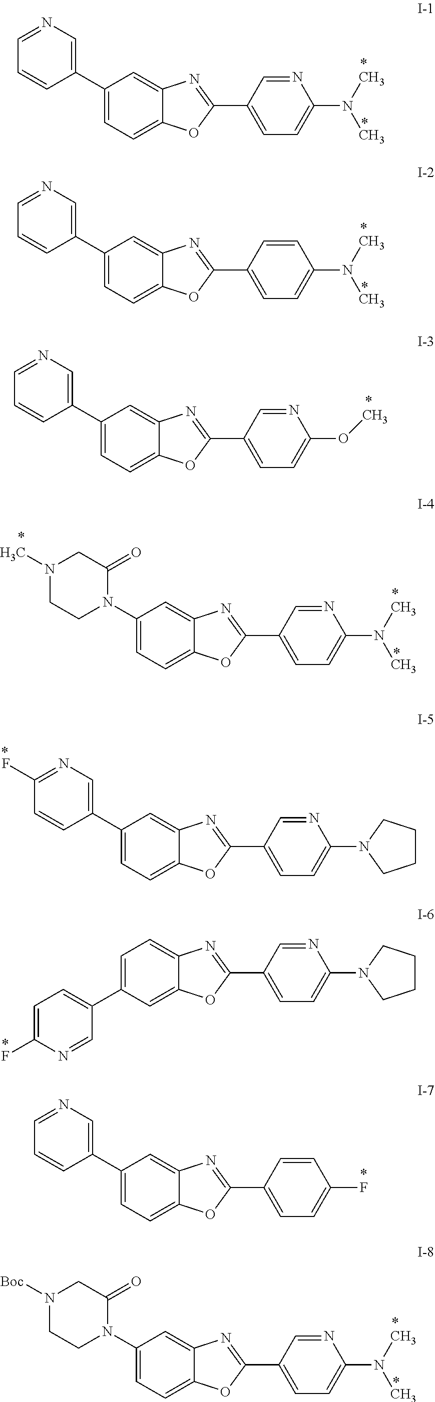

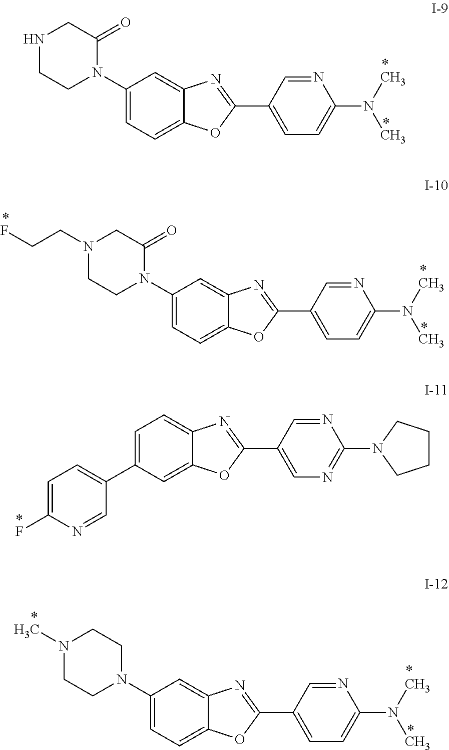

Any atom marked with * shown in the structures above may be a radioisotope of that atom, such as ¹¹C or ¹⁸F. Preferably, the *F in the compound is the radioisotope ¹⁸F, and the *C of the methoxyl or dimethylamino group attached to the aryl group is the radioisotope ¹¹C.

In this specification, the meaning of the designation as (¹⁸F) I-5 is the structure named I-5 with the * marked atom ¹⁸F; In the same way, the meaning of the named (¹¹C) I-4 means that the atom marked with * in the structure numbered I-4 is ¹¹C.

### [Composition for Optical Imaging of α-Synuclein Aggregates]

Compositions of the present invention for optical imaging of α-synuclein aggregates (also hereinafter referred to as optical imaging compositions) comprise compounds of Formula I, pharmaceutically acceptable salts thereof, or solvates thereof. Optical imaging includes biological imaging in vitro and in vivo, which include but are not limited to the fluorescence microscopy method, multi-photon imaging method, two-photon imaging method, and near-infrared fluorescence imaging method.

### [Composition for Radiographic Imaging of α-Synuclein Aggregates]

A radiolabelled composition of the present invention for radiographic imaging α-synuclein aggregates (also hereinafter referred to as a radiographic imaging composition) comprises a radiolabelled Formula I compound, a pharmaceutically acceptable salt thereof, or a solvate thereof. Radiographic imaging includes imaging in vitro and in vivo, which include but are not limited to autoradiography, PET, and SPECT.

Compositions for optical imaging or radiographic imaging may be contained in the pharmaceutically acceptable carrier. The content of Formula I compounds contained therein, their pharmaceutically acceptable salts, or their solvates, and pharmaceutically acceptable carriers are not specified and may be adjusted according to many factors such as the compound used and age, weight, health status, sex, and content of the diet of the mammal being given: the frequency and means of giving; treatment period; and other agents used at the same time.

[Diagnostic Agents for Diseases Associated with α-Synuclein Aggregates, or Accompanying Diagnostic Agents for the Treatment or Prevention of Said Diseases]

A diagnostic drug of the invention for an α-synuclein aggregates-associated disease, or a concomitant diagnostic drug for the treatment or prevention of the disease (hereinafter also referred to as a concomitant diagnostic drug) comprises the compound of the invention. A therapeutic concomitant diagnostic drug is a diagnostic drug used to determine whether a treatment is likely to be implemented after identifying the disease. In addition, prophylactic diagnostic drugs refer to a diagnostic drug used to predict the future onset of the disease or to determine whether it is possible to implement preventive disease suppression after the precursor symptoms of the disease are identified.

Compare the relevant data on the amount and/or distribution of α-synuclein aggregates in the subject's body (e.g., brain) obtained by the diagnostic drug or the concomitant diagnostic drug and the previously known correlations in the disease, it enables the subject to be diagnosed with the disease (specifically, such as whether they suffer from the diseases, severity, likelihood of onset, etc.), and better understand the disease status of the subject for formulating the prevention/treatment plan (type and combination of prophylactic administration/treatment drugs, dosage, usage, etc.).

### [Optical Imaging Method]

The optical imaging method of the invention comprises the following steps, which are illustrated in the following example of detecting α-synuclein aggregates in the brain, and similar methods for detecting other parts.

An effective amount of the tracer of the present invention is given to the tested organism, and the tracer that reaches the brain of the organism binds to the α-synuclein aggregates in the brain. Optical imaging (imaging) of the α-synuclein aggregates is then achieved by irradiating the first wavelength of light from outside the brain used to excite the tracer and detecting the second wavelength of light (e.g. fluorescence) emitted from the intracranial tracer. Wherein, the tracer contains a compound represented by Formula I, or a pharmaceutically acceptable salt thereof, or a solvate thereof.

### [Radiographic Imaging Method]

The radiographic imaging method of the invention comprises the following steps, which are illustrated in the following example of detecting α-synuclein aggregates in the brain, and similar methods for detecting other sites.

An effective amount of radiolabelled tracer of the present invention is given to the tested organism and will bind to the α-synuclein aggregates when reaching the brain. The radiation emitted from the tracer is then detected by PET or SPECT, enabling radiographic imaging (imaging) of the α-synuclein aggregates in the brain. Wherein the tracer contains a compound represented by Formula I, or a pharmaceutically acceptable salt thereof, or a solvate thereof, wherein one or more atoms of the compound of Formula I are radioisotopes of that atom.

The optical and radiological imaging subjects include mammals such as humans, rats, mice, rabbits, guinea pigs, hamsters, monkeys, dogs, minks, or miniature pigs. Preferably, mammals are humans. The tracer can be given orally, intravenously, or peritoneally without special limitation. Intravenous or intraperitoneal injection is preferred, and intravenous injection is most preferred.

### [Method for Quantifying or Determining the Accumulation of α-Synuclein in the Brain]

By calculating the difference in the amount and/or distribution of light or radiation detected in the subject organism (e.g., brain) to the normal mammals, the accumulation of α-synuclein in the body (e.g., brain) can be quantified and the presence or absence of α-synuclein aggregates in the body (e.g., brain) can be determined.

[Screening Methods for Therapeutic or Prophylactic Drugs to Prevent or Treat Diseases Associated with α-Synuclein Accumulation]

Based on the imaging method described above of [optical imaging method] and [Radiological imaging method], the light or radiation emitted by the tested organism is detected before and after administration of the screening drug, and the change of α-synuclein accumulation is determined according to the difference in its intensity and/or distribution for screening therapeutic or preventive drugs. For example, if the amount (intensity) of light (such as fluorescence) or radiation from the tracer is reduced after administration of the screening drug, the screening drug may be potentially used as a therapeutic or prophylactic drug for the disease or symptom. Preferably, if the amount and/or distribution of light or radiation from the animal model treated with the screening drug is close to normal animals (preferably mammals), the screening drug may have a better chance of therapeutic or preventing the disease or symptom.

The type of organism tested and the method of administration are the same as described above [Optical imaging method] and [Radiological imaging method].

The compounds of the present invention can be synthesized from known materials (e.g. commercially available materials) by a known method. A person skilled in the art may appropriately select starting materials and methods to synthesize the required compounds of the invention. The invention is further described below with examples, and it is understood that these examples are intended only to illustrate the invention but not to limit the scope of protection of the invention. Experimental methods not specified in the following examples are usually performed under general conditions or as recommended by the manufacturer. The known starting material of the invention may be prepared by a method known in the field, or purchased from a commercially available product. The structure of the compound is determined by nuclear magnetic resonance spectroscopy (NMR) and/or mass spectrometry.

### Example 1: Preparation of Compound I-1, Whose Structure is Shown Below

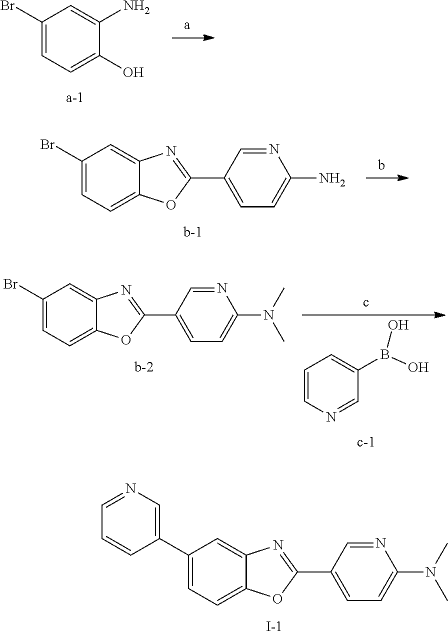

### Step a: Prepare Intermediate b-1

1.0 mmol compound 2-amino-4-bromo-phenol (a-1) was dissolved in 5 ml of methanol and added to 2.0 mmol NaOH and 1.0 mmol 2-amino-5-trifluoromethyl pyridine under nitrogen atmosphere. After heating and reflux for 10 hours, the solvent was removed and ethyl acetate was added to the residue. The product was obtained by silica gel column chromatography as a white solid with a yield of 40%. ESI-MS (positive): 290.1 (M+1)+.

### Step b: Prepare Intermediate b-2

1.0 mmol of intermediate b-1 was dissolved in 4 ml of N, N-dimethylformamide (DMF), followed by 2 mmol K₂CO₃ and 4 mmol CH₃I at room temperature for 8 h, and then extracted with ethyl acetate after adding water. After drying over anhydrous sodium sulfate, the organic phase was purified to give product b-2 as a yellow solid with a yield of 57% by silica gel column chromatography. ESI-MS (positive): 318.1 (M+1)+.

### Step c: Preparation of Compound I-1

1 mmol of b-2 was dissolved in 3 ml of N, N-dimethylformamide, and added to 2 mmol K₂CO₃, 1% Pd(PPh₃)₄, 1 mmol of 3-pyridine-boric acid (c-1) under nitrogen atmosphere. After heating and reflux for 10 hours, the mixture was added to water, extracted with ethyl acetate, and dried over anhydrous sodium sulfate. Finally, a light yellow solid was obtained by silica gel column chromatography with a yield of 30%. 1H NMR (600 MHZ, DMSO-d₆) δ 8.93 (d, J=2.2 Hz, 1H), 8.54 (d, J=4.6 Hz, 1H), 8.22-8.02 (m, 4H), 7.83 (d, J=8.2 Hz, 1H), 7.72 (d, J=8.4, 1H), 7.55-7.43 (m, 1H), 6.82 (d, J=2.2 Hz, 1H), 3.06 (s, 6H). ESI-MS (positive): 317.1 (M+1)⁺.

### Example 2: Preparation of Compound I-2 Whose Structure is Shown Below

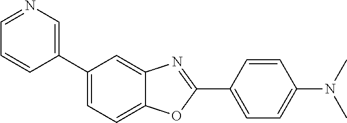 [SMILES: CN(C)c1ccc(-c2nc3cc(-c4cccnc4)ccc3o2)cc1]

The preparation method is the same as that of compound I-1, except that 2-amino-5-trifluoromethyl pyridine is replaced by 2-amino-5-trifluoromethyl benzene in step a to obtain a yellow solid with a yield of 45%, 1H NMR (600 MHZ, DMSO-d₆) δ 8.96 (d, J=2.3 Hz, 1H), 8.59 (dd, J=4.8, 1.5 Hz, 1H), 8.18-8.10 (m, 1H), 8.06-8.00 (m, 3H), 7.81 (d, J=8.3 Hz, 1H), 7.67 (dd, J=8.4, 1.9 Hz, 1H), 7.57-7.47 (m, 1H), 6.92-6.84 (m, 2H), 3.05 (s, 6H). ESI-MS (positive): 316.1 (M+1)⁺.

### Example 3: Preparation of Compound I-3 Whose Structure is Shown Below

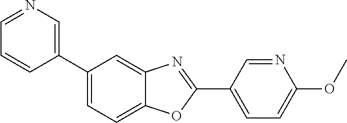 [SMILES: COc1ccc(-c2nc3cc(-c4cccnc4)ccc3o2)cn1]

The preparation method is the same as that of compound I-1, except that 2-amino-5-trifluoromethyl pyridine is replaced by 2-methoxy-5-trifluoromethyl pyridine in step a. Yellow solid was obtained with a yield of 26%. 1H NMR (600 MHZ, DMSO-d₆) δ 8.89 (d, J=2.2 Hz, 1H), 8.62 (d, J=4.6 Hz, 1H), 8.21-8.00 (m, 4H), 7.85 (d, J=8.3 Hz, 1H), 7.71 (d, J=8.4, 1H), 7.52-7.46 (m, 1H), 6.80 (d, J=2.2 Hz, 1H), 3.75 (s, 3H). ESI-MS (positive): 304.2 (M+1)⁺.

### Example 4: Preparation of Compound I-4 Shown Below

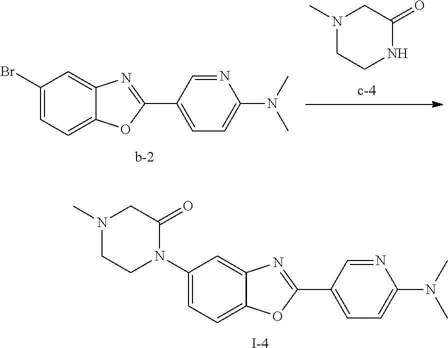

The preparation method is the same as that of the compound I-1, except that 3-pyridine-boric acid (C-1) is replaced by n-methylpiperazinone (C-4) in step c. Yellow solid with a yield of 34%. 1H NMR (600 MHZ, DMSO-d₆) δ 7.04-7.89 (m, 6H), 3.69 (s, 2H), 2.68-3.42 (m, 4H), 2.28 (s, 3H), 3.08 (s, 6H). ESI-MS (positive): 352.4 (M+1)⁺.

### Example 5: Preparation of Compound I-5, as Shown Below

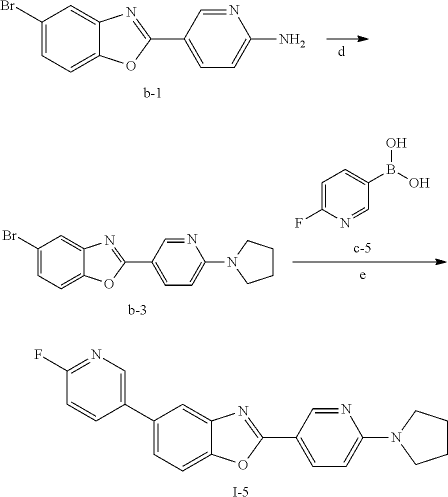

### Step d: Prepare Intermediate b-3

2.0 mmol of 1, 4-diiodobutane and 1.0 mmol of compound b-1 were dissolved in 5 ml of N, N-dimethylformamide, with 2 mmol K₂CO₃ added under nitrogen. After heated in reflux for 12 hours, the mixture was extracted with ethyl acetate after adding water. The organic phase was dried over anhydrous sodium sulfate, and a yellow solid was obtained with a yield of 8% by silica gel column chromatography. ESI-MS (positive): 344.1 (M+1)⁺.

### Step e: Preparation of Compound I-5

It is prepared by the same method as the compound I-1, except that the intermediate b-2 is replaced by the reactant b-3, and 3-pyridine-boric acid (c-1) is replaced by 2-fluoro-5-pyridine-boric acid (c-5). A light yellow solid was obtained with a yield of 42%. 1H NMR (600 MHZ, DMSO-d₆) δ 8.56 (s, 1H), 8.34 (dd, J=4.6, 1.4 Hz, 1H), 8.55 (s, 1H), 8.05 (s, 1H), 7.92-8.06 (m, 3H), 7.57 (dd, J=4.6, 1.4, 1H), 6.96-6.88 (m, 1H), 3.45-3.52 (m, 4H), 1.82-1.98 (m, 4H). ESI-MS (positive): 361.2 (M+1)⁺.

### Example 6: Preparation of Compound I-6, as Shown Below

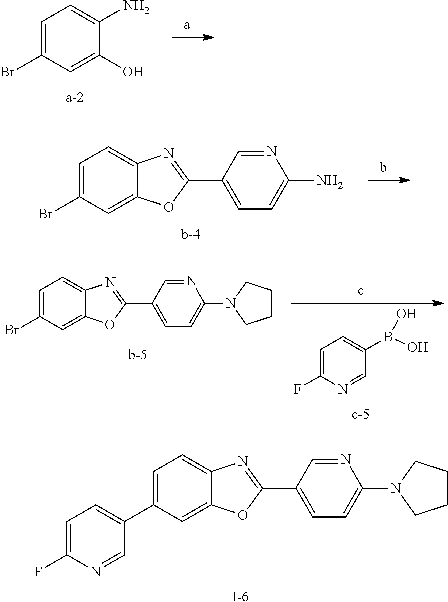

The preparation method is the same as the compound I-5, wherein the preparation of b-5 is the same as b-3, except that the starting material 2-amino-4-bromo-phenol (a-1) is replaced by 2-amino-5-bromo-phenol (a-2). A light yellow solid was obtained with a yield of 38%. 1H NMR (600 MHZ, DMSO-d₆) δ 8.51 (s, 1H), 8.28-8.8.42 (m, 1H), 8.25 (s, 1H), 7.96-8.12 (m, 3H), 7.85 (s, 1H), 7.42-7.49 (m, 1H), 6.61-6.88 (m, 1H), 3.46-3.53 (m, 4H), 1.80-1.95 (m, 4H). ESI-MS (positive): 361.2 (M+1)⁺.

### Example 7: Preparation of Compound I-7, as Shown Below

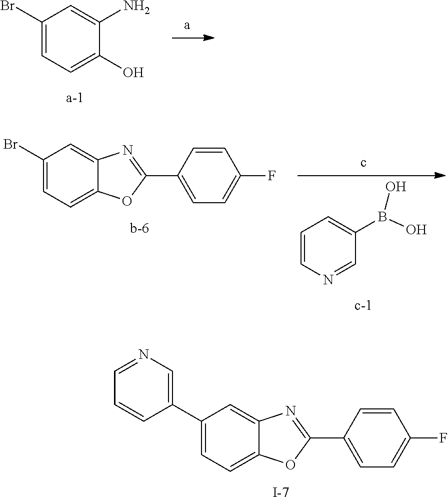

The preparation method is the same as compound I-1, except that the starting material 2-amino-5-trifluoromethyl pyridine is replaced by 4-fluoro-1-trifluoromethyl benzene in step a. A light yellow solid was obtained with a yield of 38%. 1H NMR (600 MHz, DMSO-d₆) δ 8.93 (s, 1H), 8.52-7.46 (m, 10H). ESI-MS (positive): 291.1 (M+1)⁺.

### Example 8: Preparation of Compound I-8, as Shown Below

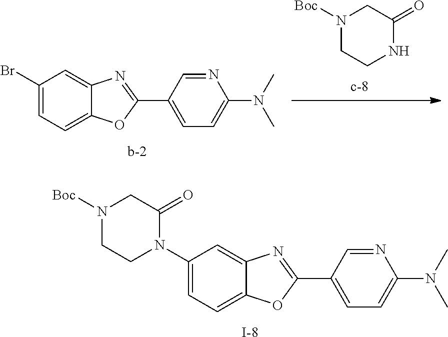

The preparation method is the same as the compound I-4, except that N-methylpiperazinone (c-4) is replaced by N-(Boc)-piperazinone (c-8). A light yellow solid was obtained with a yield of 48%. 1H NMR (600 MHZ, DMSO-d₆) δ 8.00 (d, J=8.5 Hz, 2H), 7.71 (d, J=8.7 Hz, 1H), 7.28 (d, J=8.6 Hz, 1H), 6.87 (d, J=8.6 Hz, 2H), 4.10 (s, 2H), 3.82-3.68 (m, 4H), 3.05 (s, 6H), 1.46 (s, 9H). ESI-MS (positive): 438.2 (M+1)⁺.

### Example 9: Preparation of Compound I-9, as Shown Below

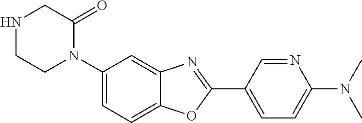 [SMILES: CN(C)c1ccc(-c2nc3cc(N4CCNCC4=O)ccc3o2)cn1]

Compound I-8 was dissolved in ethyl acetate, and then added with trifluoroacetic acid. After stirring for 12 hours, the mixture was added to water and extracted with ethyl acetate. The organic phase was dried over anhydrous sodium sulfate, and finally, a yellow solid was obtained by silica gel column chromatography with a yield of 26%. 1H NMR (600 MHZ, DMSO-d₆) δ 8.25 (s, 1H), 7.78-6.85 (m, 5H), 3.81 (s, 2H), 3.61-3.68 (m, 2H), 2.53-2.61 (m, 2H), 3.08 (s, 6H). ESI-MS (positive): 338.2 (M+1)⁺.

### Example 10: Preparation of Compound I-10, as Shown Below

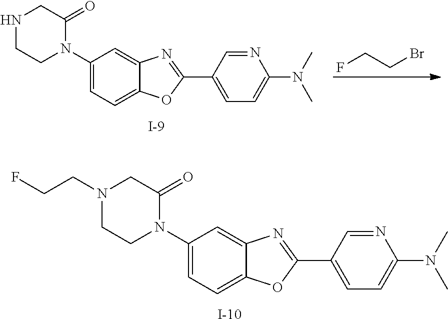

0.8 g compound I-9 was dissolved in 3 mL of N, N-dimethylformamide (DMF) with 0.08 g potassium carbonate and 0.3 g 1-bromo-2-fluoroethane, and the mixture was stirred at room temperature for 8 h. After that, 20 mL of water was added, and extracted with ethyl acetate, dried over anhydrous magnesium sulfate, and purified by silica gel column chromatography (ethyl acetate:petroleum ether=1:1) to obtain a light yellow solid with a yield of 19%. 1H NMR (600 MHZ, DMSO-d₆) δ 8.22 (s, 1H), 7.80-6.88 (m, 5H), 4.26-4.32 (m, 2H), 3.58-3.61 (m, 2H), 3.85 (s, 2H), 3.10 (s, 6H), 2.51-2.43 (m, 4H). ESI-MS (positive): 384.2 (M+1)⁺.

### [Labeling of Radionuclides]

Various radionuclides can be labelled by conventionally known methods. The preparation of (¹⁸F) I-5, (¹⁸F) I-10, and (¹¹C) I-4 is used as examples to illustrate the method of labeling ¹⁸F and ¹¹C, respectively. Other radioactive tracers can be prepared in the same way.

### Example 11: Synthesis of a Radioactive Tracer (¹⁸F) I-5

As shown in the scheme below, a variety of precursor compounds can be labelled with radionuclide ¹⁸F. The synthesis of four precursors (containing nitro, bromine, borate, or TsO-) are given below as examples but not limited to.

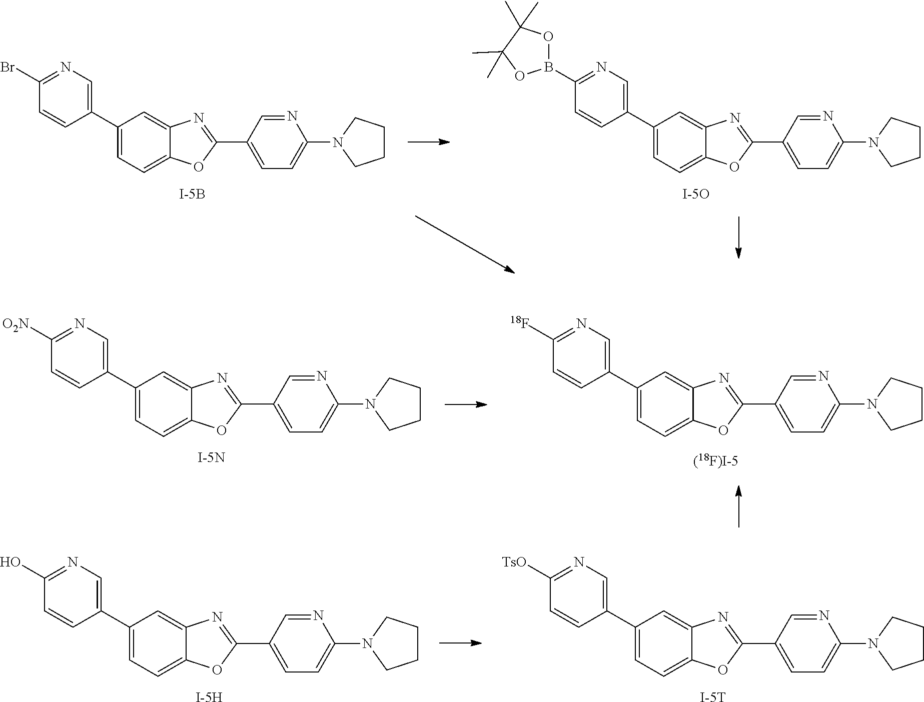

According to the method of Example 5 to replace 2-fluoro-5-pyridine boric acid with 2-nitro-5-pyridine boric acid, 2-bromo-5-pyridine boric acid, or 2-hydroxy-5-pyridine boric acid, the nitro-containing precursor compound I-5N, the bromo-containing precursor compound I-5B, and the hydroxy-containing precursor compound I-5H can be prepared, respectively. Further, the bromine-containing precursor I-5B was coupled with pinacol borate under palladium catalysis to prepare the more active precursor compound I-5O containing borate, and the hydroxyl-containing precursor compound I-5H reacted with p-methylbenzene sulfonyl chloride (TsCl) under alkaline conditions to produce the T_{S}O-containing precursor compound I-5T. All four precursor compounds of I-5B, I-5O, I-5N, and I-5T react with radioactive K¹⁸F to form the radioactive tracer (¹⁸F) I-5.

### Synthesis of a Radioactive Tracer (¹⁸F) I-5

Method 1: Synthesis from the precursor compound I-5O with borate group. ¹⁸F is produced by a cyclotron and eluted into the reaction tube by K₂₂₂/K₂CO₃ elution from bottle 1 after QMA adsorption, and evaporated in a nitrogen atmosphere at 116° C. The solution in bottle 2 (2 mL acetonitrile) was injected into the reaction tube, and the water was removed by azeotrope evaporation in a nitrogen atmosphere at 116° C. After cooling of reaction tube for 60 s, the solution in bottle 3 (8 mg precursor compound I-5O dissolved in 1 mL of DMF) was injected into the reaction tube at 115° C. and kept for 30 min. After cooling for 100 s (≤40° C.), the solution in bottle 4 (10 mL of distilled water) was injected into the reaction tube for dilution, then transferred to the C-18 column followed by eluted with 2.5 mL anhydrous ethanol, which was diluted with normal saline to less than 10% ethanol. The (¹⁸F) I-5 solution for injection was finally obtained by filtration with a 0.22 μm filter membrane. The success of the radio-labeling was proved by comparing the consistency of the retention time between the prepared radio-labelled compound and the non-radioactive compound I-5 by HPLC.

Method 2: Synthesis from the nitro-containing precursor compound I-5N. The (¹⁸F) fluoride ions were dissolved into a 50% acetonitrile solution (0.4 mL) containing K₂₂₂ (Kryptofix 222) (7.5 mg) and potassium carbonate (2.77 mg). The solution was introduced into the reaction vessel and heated under nitrogen to dry. Then anhydrous acetonitrile (0.1 mL) was added for azeotropic distillation to dry the reaction vessel fully. A DMSO (300 μL) solution containing the nitro-containing precursor compound I-5N (1 mg) was added to the reaction vessel and heated at 110° C. for 10 minutes. After cooling, (¹⁸F) I-5 was purified by HPLC.

Similarly, the brominated precursor I-5B and precursor I-5T with TsO-group can also be labelled with ¹⁸F to synthesize (¹⁸F) I-5 under similar conditions in method 2.

### Example 12: Synthesis of Radioactive Tracer (¹⁸F) I-10

As shown in the scheme below, the radioactive tracer (¹⁸F) I-10 can be prepared by direct nitro alkylation of I-9 with the bromo-alkane ¹⁸F—CH₂CH₂—Br which has been labelled by ¹⁸F. Or I-9 reacted with 1, 2-bis (toluene sulfonyloxy) ethane to produce precursor compound I-10T containing leaving group TsO-: I-9 can also be reacted with ethylene oxide to form compound I-100 containing a terminal hydroxyl group, following reacted with p-methylbenzene sulfonyl chloride (TsCl) or methane sulfonyl chloride (MsCl) under alkaline conditions to form a precursor compound (such as I-10T) with label site containing a leaving group (such as TsO- or MsO-). Finally, I-10T reacted with radioactive K₁₈F to form a radioactive tracer (¹⁸F) I-10.

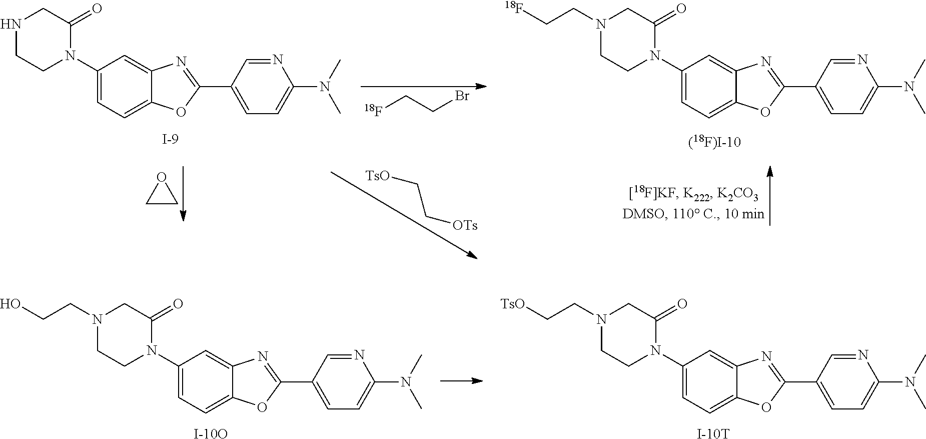

An example of the preparation of the precursor compound I-10T is shown below: 0.16 g compound I-9 dissolved in 5 mL of N, N-dimethylformamide with 0.11 g potassium carbonate (2 eq) and 0.3 g compound 1, 2-bis (toluene sulfonoxyl) ethane (2 eq). After being stirred overnight at room temperature, the mixture was added to 20 mL water and extracted with ethyl acetate. The organic phase was dried over anhydrous magnesium sulfate and the crude product was purified by silica gel column chromatography (PE:EA=1:1).

The following is an example of the preparation of (¹⁸F) I-10 from the precursor compound I-10T containing the TsO-group.

The (¹⁸F) fluoride ions were dissolved into a 50% acetonitrile solution (0.4 mL) containing K₂₂₂(Kryptofix 222) (7.5 mg) and potassium carbonate (2.77 mg). The solution was introduced into the reaction vessel and heated under nitrogen to dry. Then anhydrous acetonitrile (0.1 mL) was added for azeotropic distillation to dry the reaction vessel fully. A solution of DMSO (300 μL) dissolved with precursor I-10T (1.0 mg) was added to the reaction vessel and heated at 110° C. for 10 minutes. After cooling, (¹⁸F) I-10 was isolated and purified by HPLC (C-18 column).

### Example 13: Synthesis of a Radioactive Tracer (¹¹C) I-4 is Performed as Shown in the Scheme Below

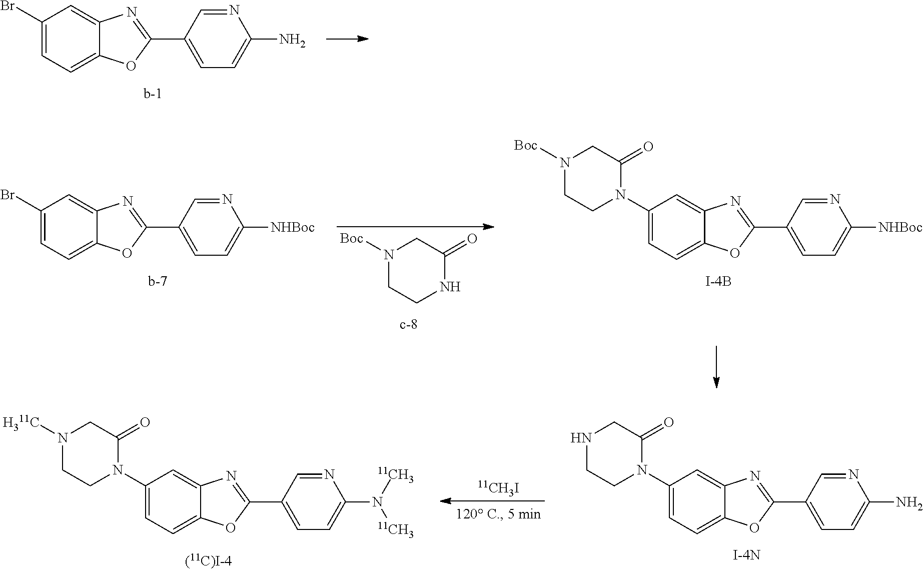

The amino group of intermediate b-1 was protected by Boc to obtain b-7, and used to synthesize I-4B in a similar method to Example 4, and then the precursor compound I-4N for radio labeling was obtained after removing the Boc protection group according to a similar method of Example 9.

The following operations were performed away from light. (¹¹C) Iodine methane was added to 300 μL of dimethyl sulfoxide (DMSO) solution dissolved with I-4N (2 mg) at room temperature. After the reaction mixture was heated at 120° C. for 5 minutes, the mixture was cooled and purified by HPLC. The component of (¹¹C) I-4 was recovered into a flask containing ethanol (300 μL), 25% ascorbic acid (100 μL), and Tween 80 (75 μL). The solvent was removed by vacuum distillation. The residue was dissolved in normal saline (3 mL, pH 7.4) to obtain (¹¹C) I-4 as an injection solution.

### [Binding Affinity of the Compounds to α-Synuclein Aggregates]

The binding affinity of the compound of the invention to human α-synuclein aggregates is determined by the fluorescence method described below.

### (1) Preparation of α-Synuclein Monomer

1 μL ampicillin anti-plasmid with α-synuclein in the correct sequence was mixed with 100 μL BL21 (DE3) receptive cells, cooled in an ice bath, added in 600 μL LB medium, and cultured in a 220 rpm shaking bed at 37° C. for 90 min. 150 μL cultured bacterial solution was added to the sterilized dish coated with ampicillin medium evenly. Positive clonal colonies were selected and added to the configured ampicillin medium and cultured in an incubator at 37° C. The cultured positive clones were poured into 1 L of 2×YT medium and cultured in a 220 rpm shaker at 37° C. to make OD 600 0.6 and then cooled to 18° C. Each medium was induced to culture for 16 hours by adding 1 ml of IPTG (500 mM).

The bacteria were centrifuged at high speed for 30 min after ultrasonic. The supernatant was collected to remove DNA and hetero-proteins by Ni-NTA chromatography. α-synuclein monomer was purified by molecular exclusion chromatography and the purity was verified by SDS-PAGE discontinuous electrophoresis.

### (2) Preparation of α-Synuclein Aggregates

α-synuclein monomers were prepared into a buffer solution containing 1×PBS, wherein the final protein concentration was 100 μM (about 5 mg/mL), and incubated in a 1000 rpm shaker at 37° C. for 7 days to obtain α-synuclein aggregates. The initial and final concentrations of the monomer protein were determined by the BCA method.

### (3) Binding Activity Test of the Compound

10 mM mother solution of DMSO was prepared from 1 mg of the compound, then diluted to 20 μM with PBS, and made gradient dilution 7 times (3× each dilution); 30 μL of test compound was added to the 384-well plate, following the experimental group was added to 30 μL α-synuclein aggregates (3 μM), and the control group was added to the same amount of PBS. After the 384-well plate was incubated shaking at 50 rpm at room temperature for 1 hour, the plate was taken out to detect the maximum absorption and emission wavelengths of the compound with an ELISA Microplate Reader, and the fluorescence values were detected at these wavelengths, too. The fluorescence changes in different concentrations of compounds were calculated using the experimental group data minus the control group data, and the protein-binding affinity of compounds was obtained using the Saturation binding module of GraphPad Prism.

The binding affinity of the Formula I compound to α-synuclein aggregates is determined by the above method, and the dissociation equilibrium constant K_{d} is shown in Table 1.

TABLE 1
Binding affinity (K_{d}) of partial Formula I compound
of the invention on human α-synuclein aggregates

| Example | K_{d} | Example | K_{d} | Example | K_{d} |
|---|---|---|---|---|---|
| compound | (μM) | compound | (μM) | compound | (μM) |
| I-1 | * | I-2 | * | I-4 | *** |
| I-5 | ** | I-6 | ** | I-10 | ** |

*** 0.1~0.2 μM;
** 0.2~0.5 μM;
* 0.5~1.0 μM

### [Optical Imaging in Patient's Brain]

Staining and Imaging of Brain Slices from Patients of Dementia with Lewy Body (DLB)

Brain slices from patients with dementia with Lewy bodies were taken from the amygdala of a deceased 75-year-old man in stage 2 of dementia with Lewy bodies. Frozen sections of the amygdala with rich α-synuclein were performed with a thickness of 20 μm.

The tested compound was diluted to 30 μM with PBS solution containing 50% EtOH, following incubated with the fresh frozen brain sections at room temperature for 30 minutes, then washed with 50% ethanol solution for 5 minutes and ultra-pure water twice for 3 minutes each time in sequence. After the sections were buried with an embedding agent (VECTASHIELD H-1000, Vector Laboratories), images of the lesion accumulation area on the sections were obtained by fluorescence microscopy. Analysis software (Image J) was used to quantify the fluorescence radiance of both the lesion area and the area of the non-forming lesion (background) to evaluate binding selectivity.

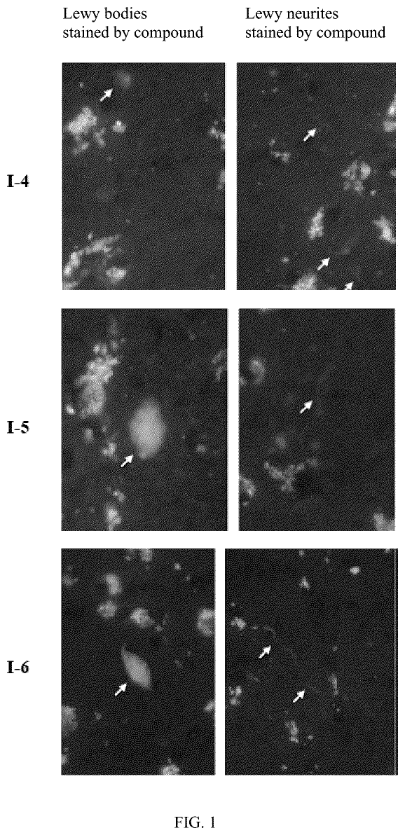

Results of the fluorescent image showed that compounds of I-4, I-5, and I-6 can stain Lewy bodies and Lewy neurites in brain slices of patients with Lewy body dementia (FIG. 1), indicating strong binding affinity to α-synuclein lesions in the brain of patients.

Staining and Imaging of Brain Slices from Patients with Alzheimer's Disease (AD)

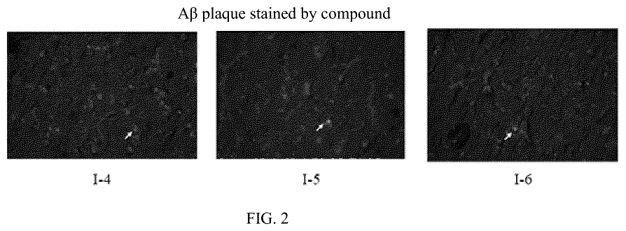

Brain slices of the superior temporal gyrus were taken from a stage 3 Alzheimer's patient after the death. The dewaxed brain tissue was fixed in a 10% neutral buffer of formalin solution, embedded with paraffin, and then sliced with a thickness of 6 μm. The staining method is the same as the above method for DLB patients. As shown in FIG. 2, the fluorescence image results showed that compounds of I-4, 1-5, and I-6 could also detect Aβ lesions but fail to Tau neurofibrillary filaments. The staining signal of the compounds was much weaker in AD brain slices than in DLB brain slices, indicating that the binding affinity of I-4, I-5, and I-6 to both Aβ and Tau histology was very weak.

According to the results of FIG. 1 and FIG. 2, the binding of compounds of I-4, I-5, and I-6 to α-synuclein pathological tissue was significantly stronger than that to Aβ and Tau pathological tissue, indicating their good binding selectivities for α-synuclein aggregates.

### [Blood-Brain Barrier Permeability Test]

The compound of the invention is injected into a rat tail vein to determine the blood-brain barrier permeability in vivo according to the following method.

Dissolve the tested compound in DMSO, and add castor oil and PBS for dilution (DMSO:castor oil:PBS=1:1:8). SD rats were weighed and given 5 mg/kg in the tail vein. 500 μL of blood was taken 20 min after administration after anesthetized with isoflurane. Cardiac perfusion was performed with 200 mL PBS and stopped until the organ faded, following brain tissue was got out and washed with PBS.

After centrifuging the extracted blood at 9000 rpm for 5 min, 200 μL supernatant was taken and added 800 μL methanol. After centrifuging at 14000 rpm for 10 min, the supernatant was taken and filtered through a 0.22 μm filter membrane and stored at −80° C. for use.

2 mL PBS and 2 mL methanol were added to about 0.5 g of brain tissue to make a tissue homogenization, and then 1 mL homogenate was taken out and centrifuged at 14,000 rpm for 10 min after 2 mL methanol was added, 1 mL supernatant was taken through 0.22 μm filter membrane and stored at −80° C. for use. The concentration of compounds in the above supernatant of blood and brain homogenate were checked by LC-MS/MS, respectively.

A brain/blood ratio of <0.1, 0.1-0.3, or >0.3 indicates blood-brain barrier penetration of weak, moderate, or good, respectively. The test results show that the brain/blood ratio of the compounds I-4, I-5, and I-6 of the invention is close to or greater than 1.0, proving their good blood-brain barrier permeability. Since all the compounds of the invention have similar structures and their clogP values are mostly between 1.0 and 3.0, it can be predicted that other compounds of the invention should also have acceptable blood-brain barrier permeability.

### INDUSTRIAL PRACTICALITY

Because of good binding/staining affinity to α-synuclein aggregates, the compounds of the present invention and its composition are extremely important for the early detection, treatment, and prevention of severe diseases such as Parkinson's disease, which is one of the most important medical difficulties at present. The compound of the invention can be used as an imaging tracer to visualize the accumulation of α-synuclein, thus providing early diagnosis and disease progression information for various neurodegenerative diseases, such as Parkinson's disease (PD), Parkinson's disease dementia (PDD), dementia with Lewy body (DLB), multiple system atrophy (MSA), etc.

1. A compound represented by general Formula I, its pharmaceutically acceptable salt or solvate, which can be used as a tracer for the imaging diagnosis of α-synuclein accumulation diseases, 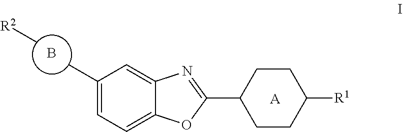
wherein,
Ring A is selected from benzene, pyridine, and pyrimidine;
R¹ is selected from 4-6 membered nitrogen-containing cycloalkyl, amino group substituted with N, N-diC₁₋₃ alkyl, C₁₋₃ alkoxyl, nitro, halogen;
Ring B is selected from pyridine, piperazine, piperazinone;
R² is selected from halogen, hydroxyl, C₁₋₃ alkyl, C₁₋₄ alkoxyl, and halogenated C₁₋₄ alkoxyl;
wherein the halogen is selected from fluorine, bromine, or iodine.

2. A compound, its pharmaceutically acceptable salt or solvate thereof according to claim 1, wherein R¹ is preferably tetrahydropyrrole, N, N-dimethylamino, or methoxyl.

3. A compound, its pharmaceutically acceptable salt or solvate thereof according to claim 1, wherein one or more atoms of the compound of Formula I are the radioisotopes of that atom, of which preferably taken from ¹¹C, ¹³N, ¹⁵O, ¹⁸F, ⁷⁶Br, ¹²³I, ¹²⁵I, and ¹³¹I.

4. A compound, its pharmaceutically acceptable salt or solvate thereof according to claim 3, wherein the compound represented by Formula I is selected from the following structures: 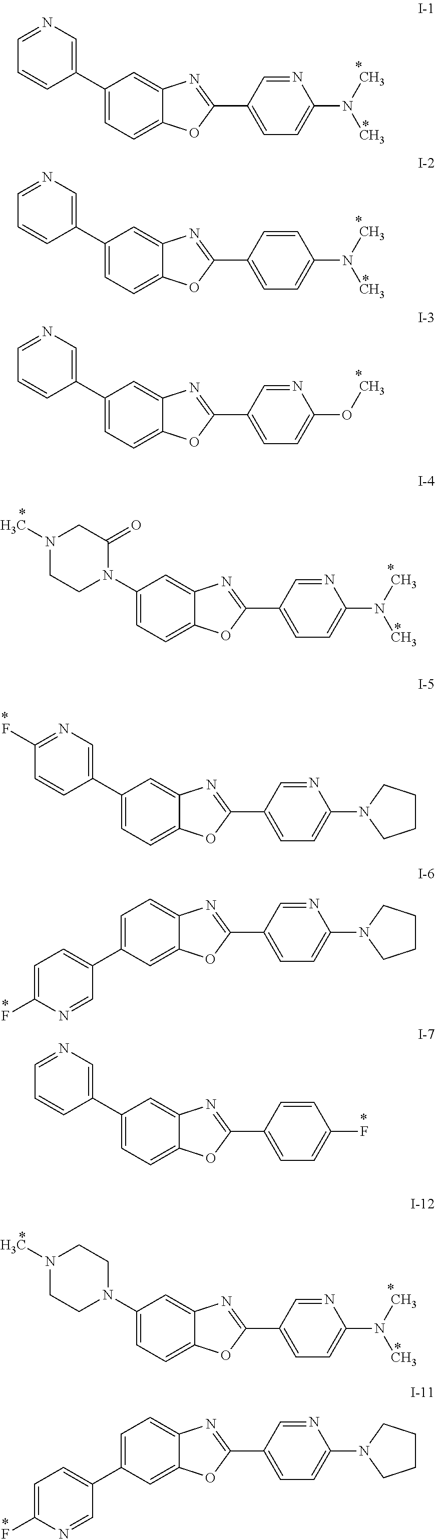
wherein one of the atoms marked with * is a radioisotope of that atom at least.

5. A compound selected from the following structures, which is used as a precursor for the synthesis of the compound according to claim 4, 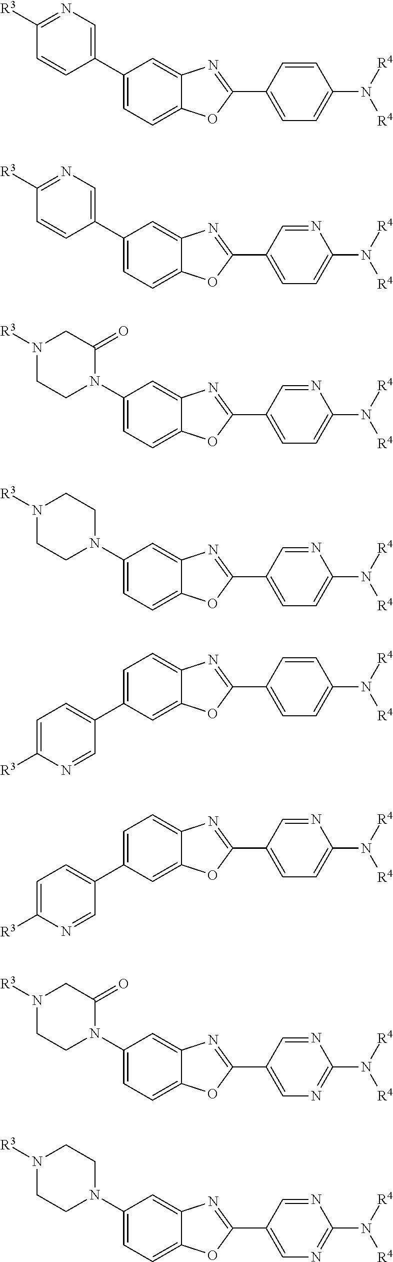 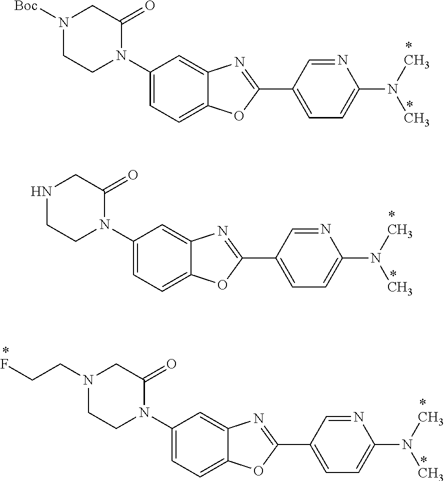
wherein, R³ is independently selected from hydroxyl, fluorine, bromine, iodine, nitro, borate group, TsO-(CH₂)ₘ—, MsO-(CH₂)ₘ—, wherein m is an integer from 0 to 4; R⁴ is independently selected from hydrogen, C₁₋₃alkyl, and 4-6 membered nitrogen-containing cycloalkyl by cyclization of NR⁴R⁴, wherein one of the atoms marked with * is a radioisotope of that atom at least.

6. (canceled)

7. (canceled)

8. The use of a compound, pharmaceutically acceptable salt, or solvate thereof according to claim 1, wherein a compound represented by Formula I or its pharmaceutically acceptable salt or solvate thereof can bind to α-synuclein aggregates and used as a tracer to image the diseases caused by α-synuclein accumulation.
